(19) United States
(12) Patent Application Publication
(10) Pub. No.: US 2010/0292463 A1
(43) Pub. Date: Nov. 18, 2010

(54) PROCESS FOR PRODUCTION OF B-LACTAM COMPOUND

(76) Inventors: Shoji Watanabe, Osaka-shi (JP); Takashi Tsukimura, Oita-ken (JP)

Correspondence Address: WENDEROTH, LIND & PONACK, L.L.P., 1030 15th Street, N.W.,, Suite 400 East, Washington, DC, 20005-1503, US

(21) Appl. No.: 12/864,296

(22) PCT Filed: Jan. 22, 2009
(86) PCT No.: PCT/JP2009/050942

§ 371 Date: Jul. 23, 2010

(30) Foreign Application Priority Data
Jan. 25, 2008 (JP) 2008-014760

(51) Int. Cl. C07D 487/04

(52) U.S. Cl. 540/350

(57) ABSTRACT

Disclosed is a process for producing a carbapenem compound represented by Formula [1], which is characterized by allowing a base and a Lewis acid metal salt to coexist in the reaction of a compound represented by Formula [2] with a compound represented by Formula [3]. According to the process, a side-chain mercaptothiazole can be introduced into a β-lactam skeleton efficiently in the production of a β-lactam compound having an excellent antibacterial activity against a Gram-positive bacterium. [3] [2] [1] wherein R¹ represents a lower alkyl group or the like; R² represents hydrogen atom or the like; R³ represents a protective group for a carboxyl group; L represents an active ester of a hydroxy group; R⁴ represents hydrogen atom or the like; and R⁵ represents hydrogen atom or the like.

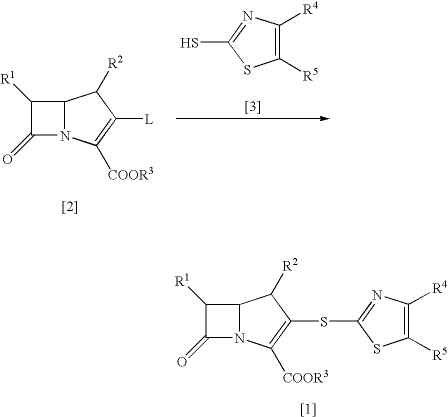

### TECHNICAL FIELD

The present invention relates to a novel process for production of a β-lactam compound represented by the generic formula [1] shown below.

### BACKGROUND OF ART

By the wide clinical application of the third-generation cephalosporins, Gram-positive bacteria have become to be frequently isolated. Particularly methicillin-resistant staphylococcus aureus (hereinafter, abbreviated as MRSA) has been more frequently isolated, and becomes a serious problem in clinical field, because infectious diseases caused by MRSA are difficult to be treated. Although vancomycin has been broadly used for infectious diseases caused by MRSA in these days, it has a defect in difficulty of administration because of its side effects, and further glycopeptide-resistant bacteria are supposed to increase in future by administration thereof. Moreover, it has recently been reported increase in frequency of isolation of methicillin-resistant and coagulase-negative staphylococci (MRCNS). Under these circumstances, it has been desired to develop a safer medicament having excellent anti-MRSA and anti-MRCNS activities.

It has been recently reported in non-patent documents 1 and 2, and patent document 1 and the like that β-lactam compounds having a thiazole nucleus at a side chain show an excellent activity against Gram-positive bacteria, especially MRSA and MRCNS. It is required to introduce mercaptothiazole into β-lactam nucleus in order to prepare these compounds. However, as described in above documents, the introduction of mercaptothiazole itself is difficult due to weakness of its reactivity. Therefore, according to the known method, step by step-reactions such as after previous activation of the mercapto group by salt formation with sodium or lithium by using a strong base such as sodium hydride (non-patent document 1) or lithium hexamethyldisilazide (example 1 of patent document 1) and the like, the activated compound is reacted with a β-lactam compound are required, and the procedures are complexed. As β-lactam compounds are unstable under a strong base, it is required to strictly control the amount of the base used in order to reduce the excessive amount of the base. Furthermore, it is necessary to protect water and the like to prevent inactivation due to the hydrolysis of the activated salt-formed compound, and therefore the method is insufficient from the viewpoint of handling and yield. Accordingly it has been desired to establish the preparation method which is easily prepared in good yield under a mild condition in case of mass-production thereof.

On the other hand, it is reported in non-patent document 3 that as an example of SH group which is poor in nucleophilicity, when ammonium dithiocarbamate is introduced into a β-lactam compound in the presence of lithium chloride, the reaction is accelerated. However there are no concrete reports therein such as procedures, yields, quality, reaction time, etc., and also no reports on its application to mercaptothiazole and use of other metal salts derived from Lewis acid therein.

As a result of studies by present inventors, there are many restrictions that lithium chloride degrades β-lactam compounds, especially the activated ester before introduction of a side chain, and even if reacting lithium chloride directly with mercaptothiazole, the reaction does not proceed, and it is also impossible to use alkylthiols which are rich in nucleophilicity, as many side-products are produced when using them.
Patent document 1: WO 02/038564 Gazette
Non-patent document 1: Bioorganic and medicinal chemistry letters, Vol. 4, p. 2793 (1994)
Non-patent document 2: Bioorganic and medicinal chemistry letters, Vol. 5, p. 1427 (1995)
Non-patent document 3: Bioorganic and medicinal chemistry letters, Vol. 7, No. 13, p. 1617 (1997)

### DISCLOSURE OF THE INVENTION

### Problem to be Solved by the Invention

The present invention is to provide effectively introducing mercaptothiazole into β-lactam nucleus as a side chain when a β-lactam compound which has an excellent antibacterial activity against Gram-positive bacteria, especially against MRSA and MRCNS is prepared.

### Means for Solving the Problem

The present inventors have studied and found that mercaptothiazole which is poor in reactivity can be effectively introduced into β-lactam nucleus by reacting them under weak basic condition in co-existence of a base and a metal salt of Lewis acid, and then the present invention was completed.

Namely the present invention is as follows:
[1] A process for preparing a carbapenem compound of the following formula [1]:

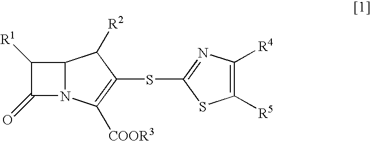

wherein R¹ is a lower alkyl group, a lower alkyl group substituted by hydroxy group or a lower alkyl group substituted by hydroxy group which is protected by a protective group, R² is hydrogen atom or a lower alkyl group, R³ is a protective group of carboxyl group, and R⁴ is hydrogen atom, an optionally substituted lower alkyl group, an optionally substituted aryl group or a group of the following formula [4]:

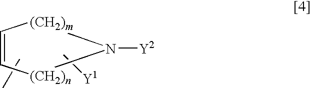

wherein m and n are independently an integer of 0 to 4 and the sum of m and n is 0 to 4, Y¹ is a halogen atom, cyano group, a hydroxy group optionally protected, an amino group optionally protected, a lower alkyloxy group, a lower alkylamino group, a carboxyl group optionally protected, a carbamoyl group optionally substituted, or a lower alkyl group optionally substituted, Y² is a hydrogen atom, a lower alkyl group optionally substituted, a lower alkenyl group optionally substituted, cyano group, a lower alkyloxycarbonyl group optionally substituted, a lower alkenyloxycarbonyl group optionally substituted, a lower alkynyloxycarbonyl group optionally substituted, an aryloxycarbonyl group optionally substituted, an aralkyloxycarbonyl group optionally substituted, a carbamoyl group optionally substituted, or —C(R⁶)═NR⁷ (wherein R⁶ and R⁷ are independently, hydrogen atom, an amino group optionally substituted or protected, or a lower alkyl group optionally substituted, or R⁶ and R⁷ may combine each other together with the carbon atom or nitrogen atom to which they bond to form a 5- to 7-membered heterocyclic group which may be substituted), provided that 1 to 4 Y¹s may present on the same ring and 2 Y¹s may present on the same carbon atom,
R⁵ is hydrogen atom, a halogen atom, cyano group, a hydroxy group optionally protected, an amino group optionally protected, a lower alkyloxy group, a lower alkylamino group, a carboxyl group optionally protected, a carbamoyl group optionally substituted, or a lower alkyl group optionally substituted, or R⁴ and R⁵ may combine each other together with the carbon atoms to which they bond to form a 5- to 7-membered saturated or unsaturated ring which may be substituted,
which comprises reacting a compound of the following formula [2]:

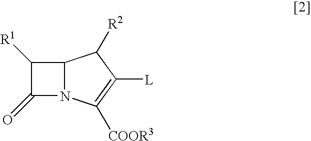

wherein L is an active ester of hydroxy group, R¹, R² and R³ are the same as defined above,

with a compound of the following formula [3]:

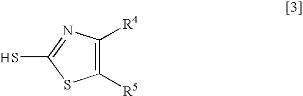

wherein R⁴ and R⁵ are the same as defined above,

characterized in that the reaction is carried out in co-existence of a base and a metal salt of Lewis acid.
[2] The process for preparing a carbapenem compound according to above item [1] wherein the base is a tertiary amine and the metal of the metal salt of Lewis acid is lithium, magnesium, calcium or aluminum.
[3] The process for preparing a carbapenem compound according to above item [1] wherein the base is a tertiary amine and the metal salt of Lewis acid is lithium chloride, lithium perfluoro C₁₋₈ alkanesulfonate, lithium bissulfonylimide represented by (Rf¹SO₂NO₂SRf²)Li (wherein Rf¹ and Rf² are the same or different, a perfluoro C₁₋₈ alkyl group or they may combine together S—N—S to which they bond to form a 5 to 7 membered ring containing perfluoroalkylene as a constitution thereof), lithium tetrafluoroborate, lithium hexafluorophosphate, lithium perchlorate, magnesium chloride, calcium chloride, or aluminum chloride.
[4] The process for preparing a carbapenem compound according to above item [1] wherein the base is a tertiary amine and the metal salt of Lewis acid is lithium chloride or magnesium chloride.
[5] The process for preparing a carbapenem compound according to above item [1] wherein the base is a tertiary amine and the metal salt of Lewis acid is lithium chloride.
[6] The process for preparing a carbapenem compound according to above item [1] wherein the base is a tertiary amine and the metal salt of Lewis acid is magnesium chloride.
[7] A process for preparing a carbapenem compound of the following formula [1]:

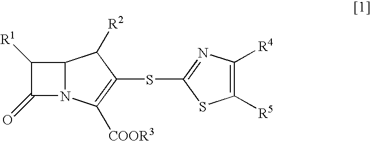

wherein R¹ is a lower alkyl group, a lower alkyl group substituted by hydroxy group or a lower alkyl group substituted by hydroxy group which is protected by a protective group, R² is hydrogen atom or a lower alkyl group, R³ is a protective group of carboxyl group, and R⁴ is hydrogen atom, an optionally substituted lower alkyl group, an optionally substituted aryl group or a group of the following formula [4a]:

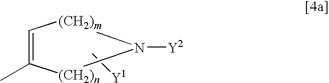

wherein m and n are independently an integer of 0 to 4 and the sum of m and n is 0 to 4, Y¹ is a halogen atom, cyano group, a hydroxy group optionally protected, an amino group optionally protected, a lower alkyloxy group, a lower alkylamino group, a carboxyl group optionally protected, a carbamoyl group optionally substituted, or a lower alkyl group optionally substituted, Y² is a hydrogen atom, a lower alkyl group optionally substituted, a lower alkenyl group optionally substituted, cyano group, a lower alkyloxycarbonyl group optionally substituted, a lower alkenyloxycarbonyl group optionally substituted, a lower alkynyloxycarbonyl group optionally substituted, an aryloxycarbonyl group optionally substituted, an aralkyloxycarbonyl group optionally substituted, a carbamoyl group optionally substituted, or —C(R⁶)═NR⁷ (wherein R⁶ and R⁷ are independently, hydrogen atom, an amino group optionally substituted or protected, or a lower alkyl group optionally substituted, or R⁶ and R⁷ may combine each other together with the carbon atom or nitrogen atom to which they bond to form a 5- to 7-membered heterocyclic group which may be substituted), provided that 1 to 4 Y¹s may present on the same ring and 2 Y¹s may present on the same carbon atom,
R⁵ is hydrogen atom, a halogen atom, cyano group, a hydroxy group optionally protected, an amino group optionally protected, a lower alkyloxy group, a lower alkylamino group, a carboxyl group optionally protected, a carbamoyl group optionally substituted, or a lower alkyl group optionally substituted, or R⁴ and R⁵ may combine each other together with the carbon atoms to which they bond to form a 5- to 7-membered saturated or unsaturated ring which may be substituted,
which comprises reacting a compound of the following formula [2]:

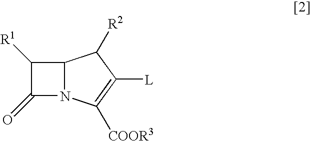

wherein L is an active ester of hydroxy group and R¹, R² and R³ are the same as defined above,

with a compound of the following formula [3]:

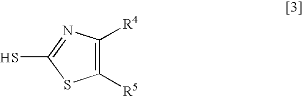

wherein R⁴ and R⁵ are the same as defined above,
characterized in that the reaction is carried out in co-existence of a base and a metal salt of Lewis acid according to any one of items [1] to [6].
[8] The process for preparing a carbapenem compound according to above items [1] to [7] wherein R⁵ is hydrogen atom.
[9] The process for preparing a carbapenem compound according to above items [1] to [8] wherein the sum of m and n is 2.
[10] The process for preparing a carbapenem compound according to above items [1] to [8] wherein the sum of m and n is 3.
[11] The process for preparing a carbapenem compound according to above items [1] to [10] wherein R⁴ is a group of the following formula [4a]:

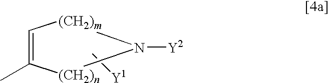

wherein m, n, Y¹ and Y² are the same as defined above; and R⁵ is hydrogen atom.
[12] The process for preparing a carbapenem compound according to above item [8], wherein R⁴ is a group of the following formula [4b]:

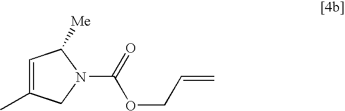

[13] The process for preparing a carbapenem compound according to above items [1] to [12] wherein R¹ is 1-(R)-hydroxyethyl or its protected group at hydroxy moiety.
[14] The process for preparing a carbapenem compound according to above items [1] to [13] wherein R² is lower alkyl group.
[15] The process for preparing a carbapenem compound according to above items [1] to [14] wherein R³ is lower alkenyl group.
[16] The process for preparing a carbapenem compound according to above items [1] to [15] wherein L is diphenylphosphate.
[17] The process for preparing a carbapenem compound according to above items [1] to [16] wherein the reaction solvent is acetone, acetonitrile, or a mixture thereof.

### Effect of Invention

According to the present invention, it became possible to effectively introduce mercaptothiazole into β-lactam nucleus as a side chain, when a β-lactam compound which has excellent antibacterial activity against Gram-positive bacteria, especially against MRSA and MRCNS is prepared.

### THE BEST MODE FOR CARRYING OUT THE INVENTION

The present invention is further explained in detail.

Number of the substituent(s) as defined as “may be substituted or is optionally substituted” or “is substituted” is not limited, if possible, and is 1 or plural. Furthermore, except for being specially indicated, the definitions in each group means a moiety of the other group or a substituent of the other group.

The lower alkyl group includes a straight chain or branched chain C₁-C₆ alkyl group, for example, methyl, ethyl, n-propyl, isopropyl, n-butyl, isobutyl, tert-butyl, n-pentyl and n-hexyl.

The lower alkenyl group includes a straight chain or branched chain C₂-C₆ alkenyl group, for example, ethenyl, 1-propeyl, 2-propenyl, 2-butenyl, 2-methyl-2-propenyl, 2-pentenyl and 3-hexenyl.

The lower alkyl group substituted by a hydroxy group includes ones having 1 to 6 carbon atoms, for example, hydroxymethyl, 1-hydroxyethyl, 2-hydroxyethyl, 1-hydroxy-1-methylethyl, 1-hydroxypropyl, and 2-hydroxypropyl.

The lower alkyloxy group includes a straight chain or branched chain C₁₋₆ alkyloxy group, for example methoxy, ethoxy, n-propoxy, isopropoxy, n-butoxy, isobutoxy, tert-butoxy, n-pentoxy and n-hexoxy.

The lower alkylamino group includes mono or di-substituted amino group substituted by a straight chain or branched chain C₁₋₆ alkyl group, for example methylamino, ethylamino, n-propylamino, isopropylamino, n-butylamino, isobutylamino, tert-butylamino, n-pentylamino, n-hexylamino, methyl ethylamino, dimethylamino, diethylamino, di(n-propyl)amino, di(isopropyl)amino, di(n-butyl)amino, di(n-pentyl)amino and di(n-hexyl)amino.

The halogen atom includes fluorine atom, chlorine atom, bromine atom and iodine atom.

The 5 to 7 membered hetero ring includes, for example 3,4-dihydro-2H-pyrrole ring, 2,3,4,5-tetrahydropyridine ring, 3,4,5,6-tetrahydro-2H-azepine ring, etc.

The substituent of lower alkyl group optionally substituted includes a hydroxy group, a lower alkyloxy group, a lower alkylthio group, a lower alkylsulfinyl group, a lower alkylsulfonyl group, a lower alkylcarbonyl group, a lower alkylcarbonyloxy group, a lower alkyloxycarbonyl group, a carboxyl group, a halogen atom, cyano group, —NR⁸R⁹ (wherein R⁸ and R⁹ are independently hydrogen atom or a lower alkyl group, or R⁸ and R⁹ may be combined together with the nitrogen atom to which they bond to form a 5-7 membered ring such as pyrrolidine, piperidine, azepane, morpholine, piperazine, or a N-lower alkyl substituted piperazine), —CONR⁸R⁹ (wherein R⁸ and R⁹ are the same as defined above), —NR^{8a}COR^{9a} (wherein R^{8a} and R^{9a} are independently hydrogen atom or a lower alkyl group), —OCONR⁸R⁹ (wherein R⁸ and R⁹ are the same as defined above), —SO₂NR⁸R⁹ (wherein R⁸ and R⁹ are the same as defined above), —NR^{8a}SO₂NR⁸R⁹ (wherein R^{8a}, R⁸ and R⁹ are the same as defined above), —NR^{8a}CONR⁸R⁹ (wherein R^{8a}, R⁸ and R⁹ are the same as defined above), and —COOCH₂OCOR¹⁰ wherein R¹⁰ is a lower alkyl group). These substituents may be protected by a suitable protective group.

The lower alkylcarbonyl group includes a straight chain or branched chain C₂₋₇ alkylcarbonyl group, for example methylcarbonyl, ethylcarbonyl, n-propylcarbonyl, isopropylcarbonyl, n-butylcarbonyl, isobutylcarbonyl, tert-butylcarbonyl, n-pentylcarbonyl and n-hexylcarbonyl.

The lower alkylcarbonyloxy group includes a straight chain or branched chain C₂₋₇ alkylcarbonyloxy group, for example methylcarbonyloxy, ethylcarbonyloxy, n-propylcarbonyloxy, isopropylcarbonyloxy, n-butylcarbonyloxy, isobutylcarbonyloxy, tert-butylcarbonyloxy, n-pentylcarbonyloxy and n-hexylcarbonyloxy.

The lower alkyloxycarbonyl group includes a straight chain or branched chain C₂₋₇ alkyloxycarbonyl group, for example methyloxycarbonyl, ethyloxycarbonyl, n-propyloxycarbonyl, isopropyloxycarbonyl, n-butyloxycarbonyl, isobutyloxycarbonyl, tert-butyloxycarbonyl, n-pentyloxycarbonyl and n-hexyloxycarbonyl.

The lower alkenyloxycarbonyl group includes a straight chain or branched chain C₃₋₇ alkenyloxycarbonyl group, for example vinyloxycarbonyl, allyloxycarbonyl, 1-propenyloxycarbonyl, 3-butenyloxycarbonyl, 2-butenyloxycarbonyl, 2-pentenyloxycarbonyl and 2-hexenyloxycarbonyl.

The lower alkynyloxycarbonyl group includes a straight chain or branched chain C₃₋₇ alkynyloxycarbonyl group, for example 2-propynyloxycarbonyl, and 1,1-dimethyl-2-propynyloxycarbonyl.

The lower alkyl portion of a lower alkylthio group, a lower alkylsulfinyl group and a lower alkylsulfonyl group includes a straight chain or branched chain C₁-C₆ alkyl group, for example, methyl, ethyl, n-propyl, isopropyl, n-butyl, isobutyl, tert-butyl, n-pentyl and n-hexyl.

The substituent of “a lower alkenyl group optionally substituted”, “a lower alkenyloxycarbonyl group optionally substituted”, “a lower alkynyloxycarbonyl group optionally substituted” and “a lower alkyloxycarbonyl group optionally substituted” includes, for example a hydroxy group, a lower alkyloxy group, a lower alkylcarbonyl group, a lower alkylcarbonyloxy group, a lower alkyloxycarbonyl group, carboxyl group, a halogen atom, and cyano group.

The substituent of a carbamoyl group optionally substituted includes one or two lower alkyl groups, or the substituent together with the nitrogen atom of the carbamoyl group, may form pyrrolidine, piperidine, and azepane.

The substituent of the amino group optionally substituted includes one or two lower alkyl groups, and the amino group substituted includes pyrrolidine, piperidine, and azepane, which is formed with the nitrogen atom of the amino group.

The substituent of the 5 to 7 membered heterocyclic group optionally substituted includes, for example, a lower alkyl group, hydroxy group, a lower alkyloxy group, a lower alkylcarbonyl group, a lower alkylcarbonyloxy group, a lower alkyloxycarbonyl group, carboxyl group, a halogen atom, and cyano group.

The aralkyl group includes, for example a C₇-C₁₂ aralkyl such as benzyl, and phenylethyl.

The aralkyloxycarbonyl group includes, for example a C₈-C₁₃ aralyloxycarbonyl such as benzyloxycarbonyl, and phenylethyloxycarbonyl.

The aryloxycarbonyl group includes, for example a C₇-C₁₁ aryloxycarbonyl such as phenyloxycarbonyl.

The arylsulfonate group includes, for example a C₆-C₁₀ arylsulfonate such as benzenesulfonate.

The compound of formula [3] wherein R⁴ and R⁵ may combine each other together with the carbon atoms to which they bond to form a 5- to 7-membered saturated or unsaturated ring which may be substituted includes a compound shown in the following formula:

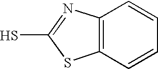

The substituent of “an aralkyl group optionally substituted”, “an aralkyloxycarbonyl group optionally substituted”, and “an aryloxycarbonyl group optionally substituted”, “an arylsulfonate group optionally substituted” and “a 5- to 7-membered saturated or unsaturated ring which may be substituted” includes, for example hydroxy group, a lower alkyloxy group, a lower alkylcarbonyl group, a lower alkylcarbonyloxy group, a lower alkyloxycarbonyl group, carboxyl group, a halogen atom, nitro group and cyano group.

The protecting group for a carboxyl group may be any conventional protecting groups, and preferably for example, a straight chain or branched chain lower alkyl group having 1 to 5 carbon atoms (e.g., methyl, ethyl, isopropyl, tert-butyl, etc.), a halogeno lower alkyl group having 1 to 5 carbon atoms (e.g., 2-iodoethyl, 2,2,2-trichloroethyl), an alkyloxymethyl group having 1 to 5 carbon atoms (e.g., methyloxymethyl, ethyloxymethyl, isobutyloxymethyl), a lower aliphatic acyloxymethyl group having 1 to 5 carbon atoms (e.g., acetoxymethyl, propionyloxymethyl, butyryloxymethyl, pivaloyloxymethyl), a 1-(C₁-C₅) lower alkyloxycarbonyloxyethyl group (e.g., 1-ethyloxycarbonyloxyethyl), a substituted or unsubstituted aralkyl group (e.g., benzyl, p-methyloxybenzyl, o-nitrobenzyl, p-nitrobenzyl), a lower alkenyl group having 3 to 7 carbon atoms (e.g., allyl, 3-methylallyl), benzhydryl group, or phthalidyl group.

The protecting group for a hydroxy group or an amino group may be any conventional ones, and preferably a lower alkyloxycarbonyl group having 1 to 5 carbon atoms (e.g., tert-butyloxycarbonyl), a halogenoalkyloxycarbonyl group having 1 to 5 carbon atoms (e.g., 2-iodoethyloxycarbonyl, 2,2,2-trichloroethyloxycarbonyl), a substituted or unsubstituted lower alkenyloxycarbonyl group having 3 to 7 carbon atoms (e.g., allyloxycarbonyl), a substituted or unsubstituted lower alkynyloxycarbonyl group having 3 to 7 carbon atoms (e.g., propargyloxycarbonyl), a substituted or unsubstituted aralkyloxycarbonyl group (e.g., benzyloxycarbonyl, p-methoxybenzyloxycarbonyl, o-nitrobenzyloxycarbonyl, p-nitrobenzyloxycarbonyl), or a trialkylsilyl group having 3 to 9 carbon atoms (e.g., trimethylsilyl, triethylsilyl, triisopropylsilyl, tert-butyldimethylsilyl), a silyl group having a phenyl group(s) (e.g. diphenylmethylsilyl, triphenylsilyl).

The preferable substituents of Y¹ on the formula [4a] of substituent R⁴ are a C₁₋₃ alkyl group such as methyl, ethyl, isopropyl, etc., hydroxymethyl, chloromethyl, fluoromethyl, methyloxymethyl, carbamoyloxymethyl (—CH₂OCONH₂), ureidomethyl (—CH₂NHCONH₂), sulfamoylmethyl (—CH₂SO₂NH₂), sulfamoylaminomethyl (—CH₂NHSO₂NH₂), carbamoyl, etc., and preferable substituents of Y² are preferably hydrogen atom, a C₁₋₃ alkyl group such as methyl, ethyl, isopropyl, etc., a C₂₋₃ alkenyl group such as ethenyl, 2-propenyl, etc., a C₃₋₇ alkenyloxycarbonyl group optionally substituted such as allyloxycarbonyl, iminomethyl (—CH═NH), —C(CH₃)═NH, etc.

The process for preparation of the present invention is explained in detail below.

A compound of the following formula [1]:

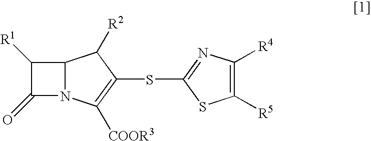

wherein R¹, R², R³, R⁴ and R⁵ are the same as defined above,

can be prepared by reacting a compound of the following formula [2]:

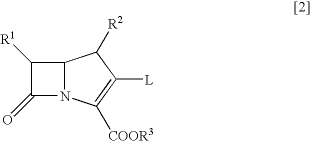

wherein R¹, R², R³ and L are the same as defined above,

with a compound of the following formula [3]:

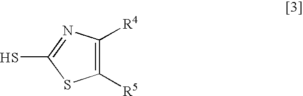

wherein R⁴ and R⁵ are the same as defined above,

in the presence of a base and a metal salt of Lewis acid.

The active ester of hydroxy group includes, for example, a substituted or unsubstituted arylsulfonic acid ester (e.g., benzenesulfonic acid ester, p-toluenesulfonic acid ester, p-nitrobenzenesulfonic acid ester, p-bromobenzene sulfonic acid ester, etc.), a lower alkanesulfonic acid ester having 1 to 5 carbon atoms (e.g., methanesulfonic acid ester, ethanesulfonic acid ester, etc.), a halogenoalkanesulfonic acid ester having 1 to 5 carbon atoms (e.g., trifluoromethanesulfonic acid ester, etc.), an arylphosphoric acid ester (e.g., diphenylphosphoric acid ester, etc.), or a halide compound such as chloride, bromide, iodide which is an ester with a hydrogen halide. The preferable reactive ester of a hydroxy group may be p-toluenesulfonic acid ester, methanesulfonic acid ester, trifluoromethanesulfonic acid ester, and diphenylphosphoric acid ester.

The inert solvent, which is used in the reaction between the compound [2] and the compound [3] in the presence of a base and a metal salt of Lewis acid to give the compound [1], includes, for example, acetone, dioxane, tetrahydrofuran, dimethyl sulfoxide, dimethylformamide, acetonitrile, benzene, toluene, hexamethylphosphoramide, or a mixture of these solvents.

The base includes, for example, a tertiary amine, etc. The tertiary amine includes an organic base such as triethylamine, diisopropylethylamine, 1,8-diazabicyclo[5.4.0]undec-7-ene (DBU). Especially preferable one is diisopropylethylamine The base should be used in an amount sufficient for carrying out the reaction, and it is usually used in a large excess amount, but it is 1 to 2 equivalents, preferably 0.8 to 1.5 equivalents to the amount of the mercaptan compound [3].

The mercaptan compound [3] should be used in an amount sufficient for carrying out the reaction, and can be used in a large excess amount, but it is usually used in an amount of 0.8 to 2 equivalents, preferably 0.8 to 1.5 equivalents to the amount of the compound [1].

The reaction is carried out at a temperature of from −78° C. to +60° C., preferably at a temperature of from −40° C. to +40° C. The reaction is different depending on the temperature, but is completed usually in 1 to 20 hours. Besides, after the reaction is completely over, the product thus obtained is isolated by a conventional technique of organic chemistry.

Metal of metal salt of Lewis acid includes lithium, magnesium, calcium aluminum, etc. Said metal salt may coordinate with water, ammonia, phosphine, etc., as well as counterion, but preferably is one without ligand such as anhydride, and the like.

Metal salt of Lewis acid includes a lithium salt, a magnesium salt, and a calcium salt, preferably a metal halide such as lithium chloride, lithium bromide, lithium iodide, magnesium chloride, aluminum chloride, or calcium chloride, lithium perfluoro C₂₋₈ alkanoate such as lithium trifluoroacetate, lithium perfluoro C₁₋₈ alkanesulfonate such as lithium trifluoromethanesulfonate, lithium pentafluoroethanesulfonate, lithium nonafluorobutanesulfonate, or lithium heptadecafluoro-1-octanesulfonate, lithium bissulfonylimide represented by (Rf¹SO₂NO₂SRf²)Li (wherein Rf¹ and Rf² are the same or different, perfluoro C₁₋₈ alkyl group or they may combine together with S—N—S to which they bond to form a 5 to 7 membered ring containing perfluoroalkylene as a component thereof) such as lithium bis(trifluoromethanesulfonyl)imido and lithium 1,1,2,2,3,3-hexafluoropropane-1,3-disulfonimido, a lithium salt of Lewis acid such as lithium tetrafluoroborate, lithium hexafluorophosphate, lithium perchlorate, and most preferably lithium chloride, or magnesium chloride.

When magnesium chloride is used, the reaction is accelerated and the obtained β-lactam compound is stable and therefore, it is preferable to use magnesium chloride. When a metal of Lewis acid is used, it is necessary for the amount enough to the reaction proceeds and an excess amount of it can be used. It is usually 0.8 to 3 equivalents to compound [1], preferably 1 to 2 equivalents.

The compounds of the generic formula [2] are known, and can be prepared by the method disclosed in JP 63-55514 B and JP 01-79180A.

The mercaptan compounds represented by the generic formula [3] are known and can be prepared starting from a known compound by the known method for example, by the method reacting hydrogen sulfide or thiourea with 2-chlorobenzothiazole, or reacting α-haloketone such as chloroacetone or phenacyl chloride with ammonium dithiocarbamate. The compounds can be also prepared by the method disclosed in WO 2002/038564.

The compound of the above mentioned formula [1] may have optical isomers based on the asymmetric carbon atoms at the 4-, 5- and 6-positions of the carbapenem nucleus, as shown in the following formula:

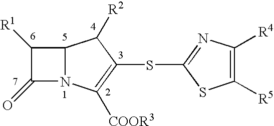

wherein R¹, R², R³, R⁴ and R⁵ are the same as defined above,

and these isomers are all conveniently expressed by only one formula.

Although the scope of the present invention should not be construed to be limited thereby and includes all isomers and a mixture of isomers based on each asymmetric carbon atom, the preferable isomers are ones wherein the 5-carbon atom has an R-configuration such as (5R,6S)-compounds when R² is a hydrogen atom, and one wherein the 4-carbon atom has an R-configuration and the 5-carbon atom has an S-configuration, such as (4R,5S,6S)-compounds, when R² is a lower alkyl group. Moreover, when R¹ is 1-hydroxyethyl group, the compound [1] may have isomers having an R-configuration or an S-configuration, as shown in the above formula, the preferable one is ones having an R-configuration.

Furthermore, there is an isomer due to a substituent, Y¹ among the substituent represented by the following formula [4] of substituent R⁴:

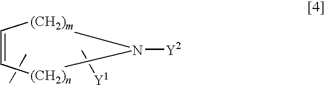

wherein n, m, n, Y¹ and Y² are the same as defined above,

Isomers having such configurations are prepared by using each corresponding isomer of the starting compounds [2] and [3].

With regard to the compound represented by following formula [1]:

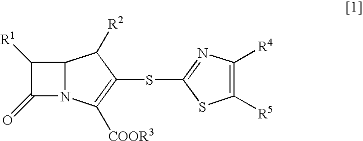

wherein R¹, R², R³, R⁴ and R⁵ are the same as defined above,

which is prepared by the method explained above,
reactions such as removal of the protecting group for hydroxy group for R¹,
removal of the protecting group of carboxyl group for R³,
and removal of the protecting group of hydroxy group, removal of the protecting group of amino group for Y¹, removal of protecting group of amino group for Y²,
(see T. W. Greene: Protective Groups in Organic Synthesis, J. Wiley & Sons Inc., 1981.) in case that R⁴ is a group represented by the following formula [4]:

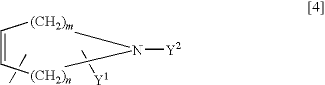

wherein m, n, Y¹ and Y² are the same as defined above,

the subsequent imidoylization reaction of the amino group of which protecting group was removed, by reacting imidates such as benzylformimidate hydrochloride or ethylacetoimidate hydrochloride (See Examples 34 and 35 of WO 2002/038564.), or the removal of protecting group (such as tert-butoxycarbonyl group, allyloxycarbonyl group, or trimethylsilyl group) of an imidoyl group for Y², are carried out, if necessary in combination of them, to give β-lactam compound or its salt which has an excellent antibacterial activity. When there are plural protecting groups which are removed with the same conditions, the removal on them can be carried out once.

A compound of the formula [1] thus obtained is subjected to deprotecting reaction on each protecting group without further purification reaction, but when a palladium catalyst such as dichlorobis(triphenylphosphine)palladium (II) and tetrakis(triphenylphosphine)palladium (0) is used for the deprotecting reaction (sulfur compounds are generally called a catalyst poison) and such impurities are contained, or unreacted materials or other impurities are contained, these impurities can be removed by extraction and the like. Especially if necessary, the compounds can be isolated and purified by a well known method such as extraction, deposition, differential chromatography, fractional crystallization, and recrystallization. The compound wherein all protecting groups are removed and the compound which is made a chemical modification such as imidoylation to promote the activity can be also taken from the reaction mixture system by a well known method, such as extraction, deposition, differential chromatography, fractional crystallization, and recrystallization to isolate or purify.

The present invention is explained by the following examples, but should not be limited by them.

The abbreviations used in Examples have the following meaning.
TMS: trimethylsilyl group
Me: methyl group
Ph: phenyl group
Tf: trifluoromethanesulfonyl group
DBU: 1,8-diazabicyclo[5.4.0]undeca-7-ene
DIEA: N-ethydiisopropylamine

### Example 1

### Allyl (4R,5S,6S)-3-({4-[(5S)-1-allyloxycarbonyl-5-methyl-2,5-dihydro-1H-pyrrole-3-yl]-1,3-thiazol-2-yl}thio)-4-methyl-7-oxo-6-[(1R)-1-trimethylsilyloxyethyl]-1-azabicyclo[3.2.0]hepto-2-ene-2-carboxylate

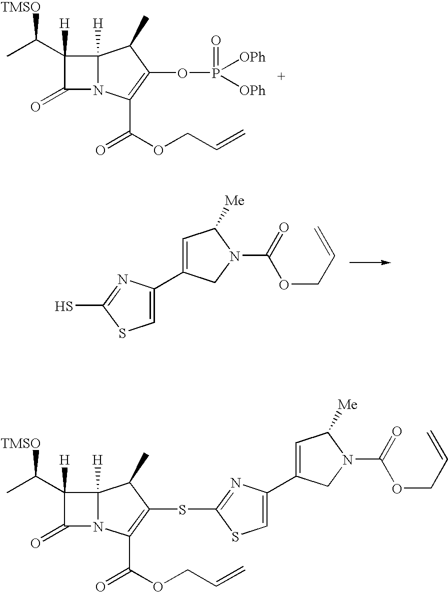

To a suspension of allyl (2S)-4-(2-mercapto-1,3-thiazol-4-yl)-2-methyl-2,5-dihydro-1H-pyrrole-1-carboxylate (0.90 g, 3.15 mmol) in acetonitrile (7.6 g) was added at room temperature diisopropylethylamine (0.41 g, 3.15 mmol). To this solution was added lithium chloride (0.25 g, 6.00 mmol), followed by stirring for 30 minutes. A solution of allyl (4R,5R,6S)-3-(diphenoxyphospholyloxy)-4-methyl-7-oxo-6-[(1R)-1-trimethylsilyloxyethyl]-1-azabicyclo[3.2.0]hepto-2-ene-2-carboxylate in acetonitrile (30%, 5.63 g, 3.00 mmol) was added at room temperature thereto, and the solution was stirred for 23 hours at the same temperature. After addition of water, the solution was extracted with ethyl acetate, and dried over anhydrous magnesium sulfate. After removal of the solvent in vacuo, the residue was purified by silica gel chromatography (hexane/ethyl acetate) to give the object compound (1.46 g, 80%).

¹H NMR (300 MHz, CDCl₃) δ 0.09 (9H, s), 1.06-1.10 (3H, m), 1.15-1.40 (6H, m), 3.21-3.24 (1H, m), 3.40-3.59 (1H, m), 4.15-4.22 (2H, m), 4.37-4.85 (7H, m), 5.15.5.48 (4H, m), 5.87.6.02 (2H, m), 6.31-6.36 (1H, m), 7.09-7.12 (1H, m).

### Example 2

### Allyl (4R,5S,6S)-3-({4-[(5S)-1-allyloxycarbonyl-5-methyl-2,5-dihydro-1H-pyrrole-3-yl]-1,3-thiazol-2-yl}thio)-4-methyl-7-oxo-6-[(1R)-1-trimethylsilyloxyethyl]-1-azabicyclo[3.2.0]hepto-2-ene-2-carboxylate

To a suspension of allyl (2S)-4-(2-mercapto-1,3-thiazol-4-yl)-2-methyl-2,5-dihydro-1H-pyrrole-1-carboxylate (0.90 g, 3.15 mmol) in acetonitrile (7.6 g) was added at room temperature diisopropylethylamine (0.41 g, 3.15 mmol). To this solution was added magnesium chloride (0.57 g, 6.00 mmol), followed by stirring for 30 minutes. A solution of allyl (4R,5R,6S)-3-(diphenoxyphospholyloxy)-4-methyl-7-oxo-6-[(1R)-1-trimethylsilyloxyethyl]-1-azabicyclo[3.2.0]hepto-2-ene-2-carboxylate in acetonitrile (30%, 5.63 g, 3.00 mmol) was added at room temperature thereto, and the solution was stirred for 3 hours at the same temperature. After addition of water, the solution was extracted with ethyl acetate, and dried over anhydrous magnesium sulfate. After removal of the solvent in vacuo, the residue was purified by silica gel chromatography (hexane/ethyl acetate) to give the object compound (1.79 g, 99%).

The compounds prepared according to the same method are shown in the following table.

TABLE 1

| | | | Metal salt of | |
|---|---|---|---|---|
| Example | Thiazole | Base | Lewis acid | Yield |
| 3 | 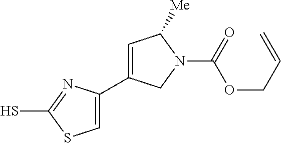 | DBU | LiCl | 86.4% |
| 4 | ↑ | None | LiCl | No reaction, degradation of substrate (starting material) |
| 5 | ↑ | DIEA | None | No reaction |
| 6 | ↑ | DIEA | LiOTf | 100% |
| 7 | ↑ | DIEA | LiBF₄ | 100% |
| 8 | ↑ | DIEA | LiPF₆ | 95.4% |
| 9 | ↑ | DIEA | Tf₂NLi | 91.0% |
| 10 | ↑ | DIEA | CaCl₂ | 92.0% |
| 11 | ↑ | DIEA | AlCl₃ | 61.8% |
| 12 | 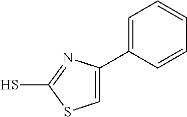 | DIEA | LiCl | 97.7% |
| 13 | 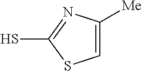 | DIEA | LiCl | 99.6% |
| 14 | 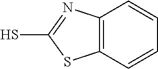 | DIEA | LiCl | 70.5% |

### INDUSTRIAL APPLICABILITY

The process for preparation of the present invention is useful for preparing a compound having excellent antibacterial activity against Gram positive bacteria, especially MRSA and MRCNS.

1. A process for preparing a carbapenem compound of the following formula [1]: 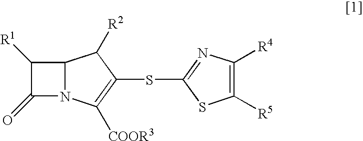
wherein R¹ is a lower alkyl group, a lower alkyl group substituted by hydroxy group or a lower alkyl group substituted by hydroxy group which is protected by a protective group, R² is hydrogen atom or a lower alkyl group, R³ is a protective group of carboxyl group, R⁴ is hydrogen atom, an optionally substituted lower alkyl group, an optionally substituted aryl group or a group of the following formula [4]: 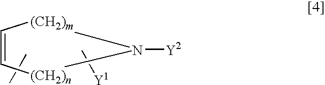
wherein m and n are independently an integer of 0 to 4 and the sum of m and n is 0 to 4, Y¹ is a halogen atom, cyano group, a hydroxy group optionally protected, an amino group optionally protected, a lower alkyloxy group, a lower alkylamino group, a carboxyl group optionally protected, a carbamoyl group optionally substituted, or a lower alkyl group optionally substituted, Y² is hydrogen atom, a lower alkyl group optionally substituted, a lower alkenyl group optionally substituted, cyano group, a lower alkyloxycarbonyl group optionally substituted, a lower alkenyloxycarbonyl group optionally substituted, a lower alkynyloxycarbonyl group optionally substituted, an aryloxycarbonyl group optionally substituted, an aralkyloxycarbonyl group optionally substituted, a carbamoyl group optionally substituted, or —C(R⁶)═NR⁷ (wherein R⁶ and R⁷ are independently, hydrogen atom, an amino group optionally substituted or protected, or a lower alkyl group optionally substituted, or R⁶ and R⁷ may combine each other together with the carbon atom or nitrogen atom to which they bond to form a 5- to 7-membered heterocyclic group which may be substituted), provided that 1 to 4 Y¹s may present on the same ring and 2 Y¹s may present on the same carbon atom, R⁵ is hydrogen atom, a halogen atom, cyano group, a hydroxy group optionally protected, an amino group optionally protected, a lower alkyloxy group, a lower alkylamino group, a carboxyl group optionally protected, a carbamoyl group optionally substituted, or a lower alkyl group optionally substituted, or R⁴ and R⁵ may combine each other together with the carbon atoms to which they bond to form a 5- to 7-membered saturated or unsaturated ring which may be substituted,
which comprises reacting a compound of the following formula [2]: 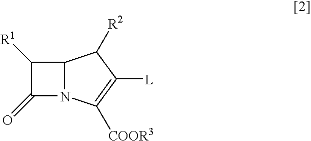
wherein L is an active ester of hydroxy group, R¹, R² and R³ are the same as defined above,
with a compound of the following formula [3]: 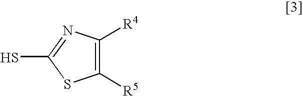
wherein R⁴ and R⁵ are the same as defined above,
characterized in that the reaction is carried out in co-existence of a base and a metal salt of Lewis acid.

2. The process for preparing a carbapenem compound according to claim 1 wherein the base is a tertiary amine and the metal of the metal salt of Lewis acid is lithium, magnesium, calcium or aluminum.

3. The process for preparing a carbapenem compound according to claim 1 wherein the base is a tertiary amine and the metal salt of Lewis acid is lithium chloride, lithium perfluoro C₁₋₈ alkanesulfonate, lithium bissulfonylimide represented by (Rf¹SO₂NO₂SRf²)Li (wherein Rf¹ and Rf² are the same or different, perfluoro C₁₋₈ alkyl group or they may combine together with S—N—S to which they bond to form a 5 to 7 membered ring containing perfluoroalkylene as a constituent), lithium tetrafluoroborate, lithium hexafluorophosphate, lithium perchlorate, magnesium chloride, calcium chloride, or aluminum chloride.

4. The process for preparing a carbapenem compound according to claim 1 wherein the base is a tertiary amine and the metal salt of Lewis acid is lithium chloride or magnesium chloride.

5. The process for preparing a carbapenem compound according to claim 1 wherein the base is a tertiary amine and the metal salt of Lewis acid is lithium chloride.

6. The process for preparing a carbapenem compound according to claim 1 wherein the base is a tertiary amine and the metal salt of Lewis acid is magnesium chloride.

7. A process for preparing a carbapenem compound of the following formula [1]: 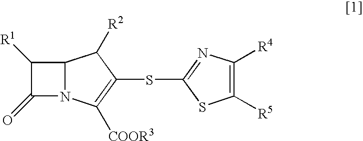
wherein R¹ is a lower alkyl group, a lower alkyl group substituted by hydroxy group or a lower alkyl group substituted by hydroxy group which is protected by a protective group, R² is hydrogen atom or a lower alkyl group, R³ is a protective group of carboxyl group, and R⁴ is hydrogen atom, an optionally substituted lower alkyl group, an optionally substituted aryl group or a group of the following formula [4a]: 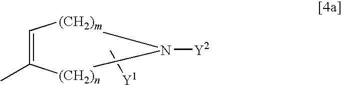
wherein m and n are independently an integer of 0 to 4 and the sum of m and n is 0 to 4, Y¹ is a halogen atom, cyano group, a hydroxy group optionally protected, an amino group optionally protected, a lower alkyloxy group, a lower alkylamino group, a carboxyl group optionally protected, a carbamoyl group optionally substituted, or a lower alkyl group optionally substituted, Y² is a hydrogen atom, a lower alkyl group optionally substituted, a lower alkenyl group optionally substituted, cyano group, a lower alkyloxycarbonyl group optionally substituted, a lower alkenyloxycarbonyl group optionally substituted, a lower alkynyloxycarbonyl group optionally substituted, an aryloxycarbonyl group optionally substituted, an aralkyloxycarbonyl group optionally substituted, a carbamoyl group optionally substituted, or —C(R⁶)═NR⁷ (wherein R⁶ and R⁷ are independently, hydrogen atom, an amino group optionally substituted or protected, or a lower alkyl group optionally substituted, or R⁶ and R⁷ may combine each other together with the carbon atom or nitrogen atom to which they bond to form a 5- to 7-membered heterocyclic group which may be substituted), provided that 1 to 4 Y¹s may present on the same ring and 2 Y¹s may present on the same carbon atom, R⁵ is hydrogen atom, a halogen atom, cyano group, a hydroxy group optionally protected, an amino group optionally protected, a lower alkyloxy group, a lower alkylamino group, a carboxyl group optionally protected, a carbamoyl group optionally substituted, or a lower alkyl group optionally substituted, or R⁴ and R⁵ may combine each other together with the carbon atoms to which they bond to form a 5- to 7-membered saturated or unsaturated ring which may be substituted,
which comprises reacting a compound of the following formula [2]: 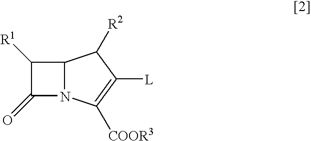
wherein L is an active ester of hydroxy group and R¹, R² and R³ are the same as defined above,
with a compound of the following formula [3]: 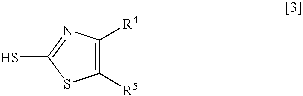
wherein R⁴ and R⁵ are the same as defined above,
characterized in that the reaction is carried out in co-existence of a base and a metal salt of Lewis acid according to claim 1.

8. The process for preparing a carbapenem compound according to claim 1 wherein R⁵ is hydrogen atom.

9. The process for preparing a carbapenem compound according to claim 1 wherein the sum of m and n is 2.

10. The process for preparing a carbapenem compound according to claim 1 wherein the sum of m and n is 3.

11. The process for preparing a carbapenem compound according to claim 1 wherein R⁴ is a group of the following formula [4a]: 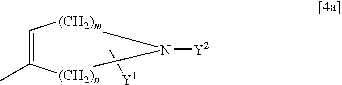
wherein m, n, Y¹ and Y² are the same as defined above; and R⁵ is hydrogen atom.

12. The process for preparing a carbapenem compound according to claim 8, wherein R⁴ is a group of the following formula [4b]:

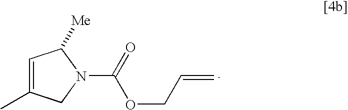

13. The process for preparing a carbapenem compound according to claim 1 wherein R¹ is 1-(R)-hydroxyethyl or its protected group at hydroxy moiety.

14. The process for preparing a carbapenem compound according to claim 1 wherein R² is a lower alkyl group.

15. The process for preparing a carbapenem compound according to claim 1 wherein R³ is a lower alkenyl group.

16. The process for preparing a carbapenem compound according to claim 1 wherein L is diphenylphosphate.

17. The process for preparing a carbapenem compound according to claim 1 wherein the reaction solvent is acetone, acetonitrile, or a mixture thereof.
